(19) United States
(12) Patent Application Publication
(10) Pub. No.: US 2021/0406687 A1
(43) Pub. Date: Dec. 30, 2021

(54) METHOD FOR PREDICTING ATTRIBUTE OF TARGET OBJECT BASED ON MACHINE LEARNING AND RELATED DEVICE

(71) Applicant: Tencent Technology (Shenzhen) Company Limited, Shenzhen (CN)

(72) Inventors: Zhi QIAO, Shenzhen (CN); Shen GE, Shenzhen (CN); Yangtian YAN, Shenzhen (CN); Kai WANG, Shenzhen (CN); Xian WU, Shenzhen (CN); Wei FAN, Shenzhen (CN)

(73) Assignee: Tencent Technology (Shenzhen) Company Limited, Shenzhen (CN)

(21) Appl. No.: 17/469,270

(22) Filed: Sep. 8, 2021

Related U.S. Application Data
(63) Continuation of application No. PCT/CN2020/086007, filed on Apr. 22, 2020.

(30) Foreign Application Priority Data
May 9, 2019 (CN) 201910386448.4

(51) Int. Cl. G06N 3/08; G06N 3/04

(52) U.S. Cl. CPC G06N 3/08; G16H 10/60; G06N 3/0427; G06N 3/0454

(57) ABSTRACT

This application discloses a method for predicting an attribute of a target object based on machine learning and a related device, which belong to the field of data prediction technologies. According to the method, a global feature of the target object is determined based on a rule feature representing historical and future change rules of a detection feature, and the global feature is refined to obtain at least one local feature of the target object, so that the refined local feature can better reflect the feature of the target object, and the attribute of the target object is further predicted based on the local feature, thereby improving the precision of the predicted attribute. When the attribute of the target object is a predicted diagnosis result, the precision of the predicted diagnosis result can be improved.

### RELATED APPLICATION

This application is a continuation application of the International PCT Application No. PCT/CN2020/086007, filed with the China National Intellectual Property Administration, PRC on Apr. 22, 2020 which claims priority to Chinese Patent Application No. 201910386448.4, filed with the China National Intellectual Property Administration, PRC on May 9, 2019, each of which is incorporated herein by reference in their entireties.

### FIELD OF THE TECHNOLOGY

This application relates to the field of data prediction technologies, and in particular, to predicting an attribute of a target object based on machine learning.

### BACKGROUND OF THE DISCLOSURE

Electronic health records (EHR) data may record every consultation record of a target object. With the development of technology, more and more clinical diagnosis estimation models may simulate a diagnosis process of a doctor based on EHR data of a patient, so as to predict a future morbidity of a user.

For example, a process of predicting the future morbidity of the user may be: using medical encoded data in the EHR data as an attribute of the patient, and inputting the medical encoded data into the clinical diagnosis estimation model, where the clinical diagnosis estimation model trains the medical encoded data, and may output a predicted diagnosis result. The process of training the medical encoded data by the clinical diagnosis estimation model may represent a diagnosis process of the doctor simulated by the clinical diagnosis estimation model, so that the future morbidity of the patient may be predicted subsequently according to the diagnosis result predicted by the clinical diagnosis estimation model.

However, due to a low accuracy of the diagnosis result predicted by the clinical diagnosis estimation model in the foregoing diagnosis prediction process, the future morbidity of the patient determined based on the predicted diagnosis result is inaccurate.

### SUMMARY

Embodiments of this disclosure provide a method for predicting an attribute of a target object based on machine learning and a related device, to resolve a problem of a low accuracy of a diagnosis result predicted by a clinical diagnosis estimation model. The technical solutions are as follows.

According to an aspect, a method for predicting an attribute of a target object based on machine learning is provided, performed by a computer device, the method including:

determining a detection feature of the target object according to detection data of the target object and an attribute corresponding to the detection data;

inputting the detection feature into a first neural network;

for a detection feature in each time series in the detection feature, outputting, by the first neural network, a first rule feature and a second rule feature different from the first rule feature through two different time series calculations, the first rule feature representing a historical change rule of the detection feature and the second rule feature representing a future change rule of the detection feature;

determining a global feature of the target object based on the first rule feature and the second rule feature;

inputting the global feature into a second neural network;

extracting and outputting, by the second neural network, at least one local feature of the target object from the global feature; and

predicting the attribute of the target object based on the at least one local feature of the target object.

According to another aspect, an apparatus for predicting an attribute of a target object based on machine learning is provided, including:

an acquisition module, configured to determine a detection feature of the target object according to detection data of the target object and an attribute corresponding to the detection data;

a calculation module, configured to input the detection feature into a first neural network; and for a detection feature in each time series in the detection feature, output, by the first neural network, a first rule feature and a second rule feature different from the first rule feature through two different time series calculations, the first rule feature representing a historical change rule of the detection feature and the second rule feature representing a future change rule of the detection feature;

the acquisition module being further configured to determine a global feature of the target object based on the first rule feature and the second rule feature;

an extraction module, configured to input the global feature into a second neural network; and extract and output, by the second neural network, at least one local feature of the target object from the global feature; and

a prediction module, configured to predict the attribute of the target object based on the at least one local feature of the target object.

According to another aspect, a computer device is provided, including: a processor; and a memory configured to store a computer program, the processor being configured to perform the computer program stored in the memory to implement the operations performed by the foregoing method for predicting an attribute of a target object based on machine learning.

According to another aspect, a non-transitory computer-readable storage medium is provided, storing a computer program, the computer program, when executed by a computer, implementing the operations performed by the foregoing method for predicting an attribute of a target object based on machine learning.

According to another aspect, a computer program product including instructions is provided, the instructions, when run on a computer, causing the computer to perform the operations performed by the foregoing method for predicting an attribute of a target object based on machine learning.

The technical solutions provided in the embodiments of this disclosure have the following beneficial effects:

The global feature of the target object is determined based on the rule feature representing the historical and future change rules of the detection feature, and the global feature is refined to obtain at least one local feature of the target object, so that the refined local feature can better reflect the feature of the target object, and the attribute of the target object is further predicted based on the local feature. Therefore, the precision of the predicted attribute can be improved. When the attribute of the target object is a predicted diagnosis result, the precision of the predicted diagnosis result can be improved.

### BRIEF DESCRIPTION OF THE DRAWINGS

To describe the technical solutions of the embodiments of this disclosure more clearly, the following briefly introduces the accompanying drawings required for describing the embodiments. Apparently, the accompanying drawings in the following description show only some embodiments of this disclosure, and a person of ordinary skill in the art may still derive other drawings from these accompanying drawings without creative efforts.

FIG. 1 is a schematic diagram of an exemplary implementation environment according to an embodiment of this disclosure.

FIG. 2 is a flowchart of a method for predicting an attribute of a target object based on machine learning according to an embodiment of this disclosure.

FIG. 3 is a schematic diagram of a diagnosis estimation model according to an embodiment of this disclosure.

FIG. 4 is a schematic structural diagram of an apparatus for predicting an attribute of a target object based on machine learning according to an embodiment of this disclosure.

FIG. 5 is a schematic structural diagram of a computer device according to an embodiment of this disclosure.

### DESCRIPTION OF EMBODIMENTS

To facilitate understanding of a method for predicting an attribute provided in this application, attribute prediction and research findings of the inventors are first described below.

A process of predicting a future morbidity of a user may be: using medical encoded data in EHR data as an attribute of a patient, and inputting the medical encoded data into a clinical diagnosis estimation model, where the clinical diagnosis estimation model trains the medical encoded data, and may output a predicted diagnosis result. The process of training the medical encoded data by the clinical diagnosis estimation model may represent a diagnosis process of a doctor simulated by the clinical diagnosis estimation model, so that the future morbidity of the patient may be predicted subsequently according to the diagnosis result predicted by the trained clinical diagnosis estimation model.

In the foregoing diagnosis prediction process, the medical encoded data is inputted into the clinical diagnosis estimation model. The medical encoded data includes data covering thousands of diseases, but one patient may suffer from one or several of the diseases only, and is unlikely to suffer from too many diseases. Therefore, useful data of the medical encoded data is distributed relatively sparsely and discretely in the medical encoded data. Moreover, the medical encoded data can only represent that the patient suffered from the disease, but cannot represent an overall physical state of the patient. In this case, after the clinical diagnosis estimation model is trained by using such medical encoded data, the accuracy of the predicted diagnosis result outputted is low, resulting in an inaccurate future morbidity of the patient determined based on the predicted diagnosis result.

To resolve the aforementioned technical problems, this application provides various embodiments for predicting an attribute of a target object based on machine learning. The method includes: determining a detection feature of the target object according to detection data of the target object and an attribute corresponding to the detection data; inputting the detection feature into a first neural network; for a detection feature in each time series in the detection feature, outputting, by the first neural network, a first rule feature and a second rule feature different from the first rule feature through two different time series calculations, the first rule feature representing a historical change rule of the detection feature and the second rule feature representing a future change rule of the detection feature; determining a global feature of the target object based on the first rule feature and the second rule feature; inputting the global feature into a second neural network; extracting and outputting, by the second neural network, at least one local feature of the target object from the global feature; and predicting the attribute of the target object based on the at least one local feature of the target object.

It can be learned that in the method for predicting an attribute of a target object based on machine learning provided in this application, the global feature of the target object is determined based on the rule feature representing the historical and future change rules of the detection feature, and the global feature is refined to obtain at least one local feature of the target object, so that the refined local feature can better reflect the feature of the target object, and the attribute of the target object is further predicted based on the local feature. Therefore, the precision of the predicted attribute can be improved. When the attribute of the target object is a predicted diagnosis result, the precision of the predicted diagnosis result can be improved.

The method for predicting an attribute of a target object based on machine learning provided in the embodiments of this disclosure is implemented based on a machine learning technology. Machine learning (ML) is a multi-disciplinary subject involving a plurality of disciplines such as probability theory, statistics, approximation theory, convex analysis, and algorithm complexity theory. The ML specializes in studying how a computer simulates or implements a human learning behavior to acquire new knowledge or skills, and reorganize an existing knowledge structure, so as to keep improving its performance. The ML is a core of the AI, is a basic way to make the computer intelligent, and is applied to various fields of the AI. The ML and deep learning generally include technologies such as an artificial neural network, a belief network, reinforcement learning, transfer learning, inductive learning, and learning from demonstrations.

In addition, machine learning is an important direction of artificial intelligence (AI). The AI is a theory, method, technology, and application system that use a digital computer or a machine controlled by a digital computer to simulate, extend, and expand human intelligence, perceive the environment, acquire knowledge, and use the knowledge to obtain the best result. In other words, the AI is a comprehensive technology of computer science, which attempts to understand essence of intelligence and produces a new intelligent machine that can respond in a manner similar to human intelligence. The AI is to study the design principles and implementation methods of various intelligent machines, to enable the machines to have the functions of perception, reasoning, and decision-making.

AI technology is a comprehensive discipline, and relates to a wide range of fields including both hardware-level technologies and software-level technologies. AI foundational technologies generally include technologies such as a sensor, a dedicated AI chip, cloud computing, distributed storage, a big data processing technology, an operating/interaction system, and electromechanical integration. AI software technologies mainly include several major directions such as a computer vision (CV) technology, a speech processing technology, a natural language processing technology, and machine learning/deep learning.

It can be learned from the foregoing content that the method for predicting an attribute of a target object based on machine learning provided in the embodiments of this disclosure relates to AI, and in particular, to the machine learning in AI.

It is to be understood that the method for predicting an attribute of a target object based on machine learning provided in the embodiments of this disclosure is applicable to a computer device capable of processing data, such as a terminal device or a server. The terminal device may include a smartphone, a computer, a personal digital assistant (PDA), a tablet computer, or the like. The server may include an application server or a Web server. During actual deployment, the server may be an independent server or a cluster server.

If the method for predicting an attribute of a target object based on machine learning provided in the embodiments of this disclosure is performed by a terminal device, the terminal device may directly predict the attribute of the target object according to the detection data of the target object inputted by a user and the attribute corresponding to the detection data, and display a prediction result for the user to view. If the method for predicting an attribute of a target object based on machine learning provided in the embodiments of this disclosure is performed by a server, the server first predicts the attribute of the target object according to the detection data of the target object uploaded by the terminal device and the attribute corresponding to the detection data to obtain a prediction result; and then sends the prediction result to the terminal device, so that the terminal device displays the received prediction result for the user to view.

To facilitate understanding of the technical solution provided in the embodiments of this disclosure, an application scenario to which the method for predicting an attribute of a target object based on machine learning provided in the embodiments of this disclosure is applicable is exemplarily described by using an example in which the method for predicting an attribute of a target object based on machine learning provided in the embodiments of this disclosure is applicable to the terminal device.

In a possible application scenario of the method for predicting an attribute of a target object based on machine learning provided in the embodiments of this disclosure, the application scenario includes: a terminal device and a user. The terminal device is configured to perform the method for predicting an attribute of a target object based on machine learning provided in the embodiments of this disclosure, and predict the attribute of the target object to obtain a prediction result for the user to view.

After receiving an attribute prediction instruction triggered by the user, the terminal device may determine a detection feature of the target object according to detection data of the target object and an attribute corresponding to the detection data; input the detection feature into a first neural network; for a detection feature in each time series in the detection feature, output, by the first neural network, a first rule feature and a second rule feature different from the first rule feature through two different time series calculations, the first rule feature representing a historical change rule of the detection feature and the second rule feature representing a future change rule of the detection feature; determine a global feature of the target object based on the first rule feature and the second rule feature; input the global feature into a second neural network; extract and output, by the second neural network, at least one local feature of the target object from the global feature; and predict the attribute of the target object based on the at least one local feature of the target object, to obtain a prediction result, so that the terminal device displays the prediction result to the user.

It is to be understood that during actual application, the method for predicting an attribute of a target object based on machine learning provided in the embodiments of this disclosure may be applicable to the server. Based on this, in another possible application scenario of the method for predicting an attribute of a target object based on machine learning provided in the embodiments of this disclosure, the application scenario includes: a server, a terminal device, and a user. After receiving an attribute prediction instruction triggered by the user, the terminal device generates an attribute prediction request according to the attribute prediction instruction, and sends the attribute prediction request to the server, so that the server may determine a detection feature of the target object according to detection data of the target object and an attribute corresponding to the detection data after receiving the attribute prediction request sent by the terminal device; input the detection feature into a first neural network; for a detection feature in each time series in the detection feature, output, by the first neural network, a first rule feature and a second rule feature different from the first rule feature through two different time series calculations, the first rule feature representing a historical change rule of the detection feature and the second rule feature representing a future change rule of the detection feature; determine a global feature of the target object based on the first rule feature and the second rule feature; input the global feature into a second neural network; extract and output, by the second neural network, at least one local feature of the target object from the global feature; and predict the attribute of the target object based on the at least one local feature of the target object, to obtain a prediction result, so that the server may feedback the obtained prediction result to the terminal device, and the user may view the prediction result on the terminal device.

It is to be understood that the foregoing application scenarios are only examples. During actual application, the method for predicting an attribute of a target object based on machine learning provided in the embodiments of this disclosure may be also applicable to another application scenario for attribute prediction. The method for predicting an attribute of a target object based on machine learning provided in the embodiments of this disclosure is not limited herein.

To make objectives, technical solutions, and advantages of this application clearer, the following further describes implementations of this application in detail with reference to the accompanying drawings.

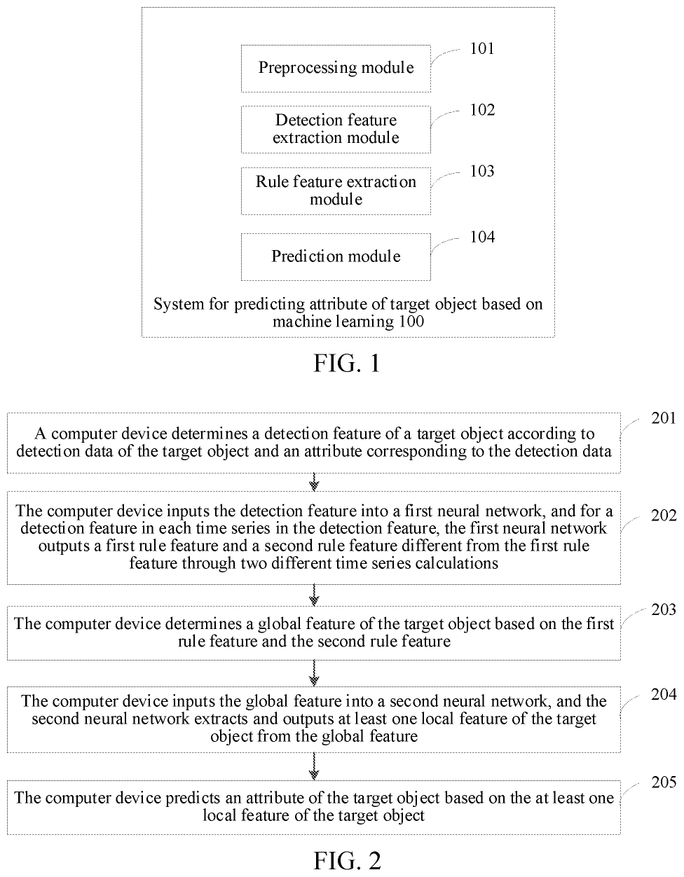

FIG. 1 is a schematic diagram of an implementation environment according to an embodiment of this disclosure. Referring to FIG. 1, the environment includes a system 100 for predicting an attribute of a target object based on machine learning. The system for predicting an attribute of a target object based on machine learning includes a preprocessing module 101, a detection feature extraction module 102, a rule feature extraction module 103, and a prediction module 104.

The preprocessing module 101 is configured to process detection data of the target object and an attribute corresponding to the detection data, and transform detection data of a user and an attribute corresponding to the detection data into data that may be calculated by the detection feature extraction module 102.

The detection feature extraction module 102 is configured to extract a mixture feature of the feature of the detection data and the attribute corresponding to the detection data, and the extracted mixture feature may be used as the detection feature of the target object. The detection feature extraction module 102 may first extract a feature of the attribute and the feature of the detection data based on the data processed by the preprocessing module 101, and then splice the feature of the attribute and the feature of the detection data that are extracted, and finally, the detection feature extraction module 102 extracts the detection feature based on a splicing result.

The rule feature extraction module 103 is configured to extract a rule feature and generate a global feature of the target object. The rule feature is used for representing a global change rule of the detection feature. The rule feature extraction module 103 may first extract a historical change rule and a future change rule based on the detection feature extracted by the detection feature extraction module 102, and the rule feature extraction module 103 may then acquire the global change rule of the detection feature based on the historical change rule and the future change rule of the detection feature, and finally determine the global feature of the target object according to the rule feature representing the global change rule.

The prediction module 104 is configured to predict the attribute of the target object. The prediction module 104 may refine the global feature generated by the rule feature extraction module 103 by using a neural network to obtain a local feature of the target object, the prediction module 104 then uses a plurality of local features expressing to acquire in the target local feature for concentration, and the prediction module 104 finally predicts the attribute of the target object based on the target local feature.

It is to be noted that functions of all the modules in the system 100 for predicting an attribute of a target object based on machine learning may be implemented by using one computer device or a plurality of computer devices, and a quantity of computer devices that implement the functions of all the modules in the embodiments of this disclosure is not limited.

FIG. 1 describes respective functions of all modules in a system for predicting an attribute of a target object based on machine learning. To reflect a specific process in which the system for predicting an attribute of a target object based on machine learning predicts the attribute, referring to FIG. 2, FIG. 2 is a flowchart of a method for predicting an attribute of a target object based on machine learning according to an embodiment of this disclosure. As shown in FIG. 2, the method for predicting an attribute of a target object based on machine learning includes the following steps:

201. A computer device determines a detection feature of a target object according to detection data of the target object and an attribute corresponding to the detection data.

The target object may be any user.

The detection data of the target object may include detection data of the target object during each detection in a historical time period, and the detection data during each detection corresponds to one detection time. Therefore, the detection data during each detection may include a plurality of types of data related to the target object. A physical sign of the target object is used as an example, data detected once (e.g., in one doctor office visit) includes heartbeat data, blood pressure data, and other types of data. For any type of data, a lot of data may be detected during each detection, and the plurality of data detected in one visit may form a time series sequence related to the detection time. Therefore, one detection time may correspond to a plurality of time series sequences, and each type of the time series sequence may be labeled to distinguish the plurality of types of detection data at one detection time. It is to be noted that the detection time is not limited in this embodiment of this disclosure, and a time interval between two detection times is not limited. In addition, the historical time period is not limited in this embodiment of this disclosure, and the historical time period may refer to any time period before the computer device predicts the attribute of the target object by using an attribute predicting method.

In this case, when the detection data is physical sign data of the target object, the detection data may be time series data stored in EHR data. The time series data includes an inquiry time of the target object during each inquiry (e.g., a doctor office visit) and physical sign data of the target object detected at each inquiry time. It is to be understood that the inquiry time is also the detection time.

The attribute is used for indicating at least one state of the target object, and each position of the attribute corresponds to one state. A state identifier may be used for indicating whether the target object has a corresponding state. The state identifier may include a first state identifier and a second state identifier. The first state identifier is used for indicating that the target object has the corresponding state, and the second state identifier is used for indicating that the target object does not have the corresponding state. For example, when any position in the attribute has the first state identifier, it indicates that the target object has the corresponding state at the position, and when any position in the attribute has the second state identifier, it indicates that the target object does not have the corresponding state at the position. In addition, different character strings may be used for representing the first state identifier and the second state identifier, and the character strings representing the first state identifier or the second state identifier are not limited in this embodiment of this disclosure.

After detecting the target object each time, detection data corresponding to this time may be obtained. Based on the detection data, the attribute of the target object may be determined, and the detection time corresponding to the determined attribute of the target object is an inspection time corresponding to the detection data at this time. It can be learned that in this embodiment of this disclosure, one attribute corresponds to one detection time, and if the detection data of the target object includes at least one detection time, the detection data of the target object corresponds to at least one attribute.

The at least one state may be a sick state of the target object or another state, and the at least one state is not limited in this embodiment of this disclosure. When the target object is in the sick state, the attribute corresponding to the detection data may be medical encoded data, and one medical encoded data may be composed of 0 and 1. Each position in the medical encoded data corresponds to one disease. When data at a particular position is 0, it indicates that the target object does not suffer from a disease at the particular position. When the data at a particular position is 1, it indicates that the target object suffers from the disease at the particular position. It is to be understood that 0 herein is equivalent to the second state identifier, and 1 herein is equivalent to the first state identifier.

To facilitate calculation, the computer device may first preprocess the detection data of the target object and the attribute corresponding to the detection data, so that the detection data and the attribute corresponding to the detection data conform to a format required by subsequent calculation; and then perform feature extraction on the processed data to obtain the detection feature of the target object. In one implementation, step 201 may be implemented through the procedure shown in step 2011 to step 2014.

Step 2011. The computer device inputs the attribute corresponding to the detection data into a fully connected neural network, screens out a target state in the attribute by using the fully connected neural network, weights the target state, and outputs a feature of the attribute corresponding to the detection data.

Because a plurality of states are stored in the attribute of the target object, the target object has some states in the attribute, and the state of the target object is used as the target state.

To facilitate calculation, the computer device preprocesses the attribute corresponding to the detection data. In one implementation, the computer device may represent the attribute corresponding to the detection data by using a multi-hot vector, to preprocess the attribute corresponding to the detection data. The multi-hot vector is composed of 0 and 1, where 0 indicates that the target object does not have the corresponding state, and 1 indicates that the target object has the corresponding state.

In this case, the computer device may input the multi-hot vector to the fully connected neural network, and the fully connected neural network screens out the target state of the target object by using an encoding matrix, and weights the screened target state to output the feature of the attribute corresponding to the detection data. In this application, the screened target state is weighted, so that a processed result may concentrate a feature of an attribute corresponding to the multi-hot vector.

Each network node in the fully connected network may calculate the data in the multi-hot vector by using a first equation. The first equation may be expressed as πⱼ=ReLU(W^{T}xⱼ+b_{π}), where W^{T} is an encoding feature matrix, and W^{T} may be a matrix trained in advance, or a matrix trained in a process in which the fully connected neural network calculates the feature of the attribute; j is a serial number of a j^{th} detection time; xⱼ is a multi-hot vector of an attribute corresponding to the j^{th} detection time, that is, xⱼ represents the multi-hot vector of the attribute corresponding to the j^{th} detection time; πⱼ is a feature vector of the attribute corresponding to the j^{th} detection time, the feature vector represents a feature of the attribute corresponding to the j^{th} detection time, and j is an integer greater than or equal to 1; and b_{π} represents a bias parameter, b_{π} and may be a parameter trained in advance, or a parameter trained in the process in which the fully connected neural network calculates the feature of the attribute.

When J represents a total quantity of detection times of the target object, after the computer device calculates all the attributes corresponding to the detection data by using the fully connected neural network, a feature π=[π₁, . . . , π_{J}] of the attribute corresponding to the detection data may be obtained. It is to be noted that the computer device may calculate the feature of the attribute by using other neural networks than the fully connected neural network.

Because one attribute is used for indicating at least one state of the target object, when the attribute indicates more states, if the computer device represents the attribute by using a multi-hot vector of the attribute, a dimension of the multi-hot vector may be relatively high. In this case, the dimension of the feature π of the attribute may be reduced in comparison to the dimension of the multi-hot vector by screening the target states, so that the process shown in step 2011 may also be regarded as a dimension reduction process, thereby facilitating subsequent calculation.

Step 2012. The computer device inputs the detection data into a time series analysis tool, extracts a feature of each type of data in the detection data in each time series by using the time series analysis tool, and outputs a feature set.

The time series analysis tool may be a highly comparative time-series (HCTSA) codebase. The feature of each type of data in each time series may include a feature representing a data distribution, entropy, and scale attribute and the like of the type of data. It can be learned that the features may represent an autocorrelation structure of the type of data. Because the features are obtained based on actual detection data, the features are interpretable.

After the computer device inputs the detection data at the j^{th} time into the time series analysis tool, the time series analysis tool may extract a feature {Sⱼ^{k}, k=1, . . . , z} of each type of data from the detection data at the j^{th} time based on a preset feature extraction rule, where Sⱼ^{k} represents a time series feature of a k^{th} data type during the j^{th} detection, z is a positive integer, k is a serial number of the data type, j represents a serial number of the j^{th} detection, and is also a serial number of the j^{th} detection time, j is a positive integer, 1≤j≤J, and J represents the total quantity of detection times of the target object. It is to be noted that the preset feature extraction rule is not limited in this embodiment of this disclosure.

If J represents the total quantity of detection times of the target object, after all the detection data of the J times is processed by using the time series analysis tool, the time series analysis tool stores the feature of the detection data extracted each time in a feature set {S₁^{k}, . . . , S_{J}^{k}, k=1, . . . , z}, and the time series analysis tool may finally output the feature set, so that the computer device may obtain the feature set.

Because the features in the feature can only represent the autocorrelation structure of all these types of data, and cannot reflect all the features of the detection data, the computer device also needs to acquire a feature of the detection data by performing the following step 2013.

Step 2013. The computer device inputs the feature set into a deep & cross neural network, and performs cross processing on a feature of each time series in the feature set by using the deep & cross neural network to output a feature of the detection data.

The deep & cross network (DCN) includes a cross network and a deep network. The computer device may separately input the feature set into the cross network and the deep network, may cross a plurality of time series features in the feature set by using the cross network, to output the crossed features, extract common features of all the features in the feature set by using the deep network, and finally combine the crossed features outputted by the DCN with the common features extracted by the deep network, to obtain the feature of the detection data.

It is to be noted that the execution sequence of step 2011 to step 2013 is not limited in this embodiment of this disclosure. For example, the computer device may first perform step 2011 and then perform step 2012 and step 2013; or first perform step 2012 and step 2013 and then perform step 2011; or may perform step 2012 and step 2013 simultaneity when performing step 2011.

Step 2014. The computer device inputs the feature of the attribute and the feature of the detection data into a deep neural network, extracts a mixture feature of the detection data and the attribute corresponding to the detection data by using the deep neural network, and outputs the detection feature.

The computer device may first splice the feature of the attribute and the feature of the detection data to obtain a spliced feature, and then input the spliced feature into the deep neural network.

In one implementation, if the feature πⱼ of the attribute corresponding to the j^{th} detection and the feature τⱼ of the detection data during the j^{th} detection are spliced into χⱼ by using a function concat[ ] for connecting arrays, χⱼ=concat[τⱼ,τⱼ]. Then, χⱼ is used as an input of the deep neural network, and each node in the deep neural network may calculate data in χⱼ according to a second equation as follows, so that the deep neural network may output a detection features ξⱼ at the j^{th} detection time. The second equation may be expressed as ξⱼ=ReLU(Wₓ^{T}χⱼ+bₓ), where Wₓ is a first weight matrix, Wₓ^{T} is a transposed matrix of Wₓ, and bₓ is a first bias parameter.

Because each weight of Wₓ is used for representing an importance degree of each element in χⱼ, each element in χⱼ may be weighted by using Wₓ^{T}χⱼ, and the elements in χⱼ may be further integrated. A rectified linear unit (ReLU) function may better mine related features between data. Therefore, the ReLU function has a relatively strong expressive ability. Wₓ^{T}χⱼ is processed by using the ReLU function, and the processed results ξⱼ may express the feature included in χⱼ. Therefore, ξⱼ may be used as the detection feature during the j^{th} detection.

When a feature of an attribute corresponding to each detection time and a feature of the detection data are spliced, the computer device may then extract a detection feature at each detection time by using the deep neural network. For ease of description, the detection feature at each detection time is referred to as a sub-detection feature. Therefore, the detection feature finally outputted by the deep neural network includes at least one sub-detection feature. The computer device may store the at least one sub-detection feature in the detection feature according to a time series to obtain a detection feature ξ=[ξ₁, ξ₂, . . . ξ_{J}], where ξⱼ is a j^{th} sub-detection feature, and is also the detection feature corresponding to the j^{th} detection time, j is a positive integer, 1≤j≤J, and J represents a total quantity of detection times of the target object.

Because the feature of the detection data and the feature of the attribute include the features of various types of data in each time series, the detection feature has multi-modes, so the detection feature may be regarded as a multi-mode feature.

Because the detection feature is obtained based on the detection data and the attribute corresponding to the detection data, compared with the feature of the target object obtained only based on the detection data, the detection feature in this embodiment of this disclosure can better reflect the feature of the target object during detection. Moreover, the detection data is data detected from actual practice rather than generated data, and may be used as an objective basis, so that the obtained detection feature is interpretable, and the attribute corresponding to the detection data is a result of a subjective judgment, so that the precision of the detection feature obtained based on the attribute and the detection data is relatively high.

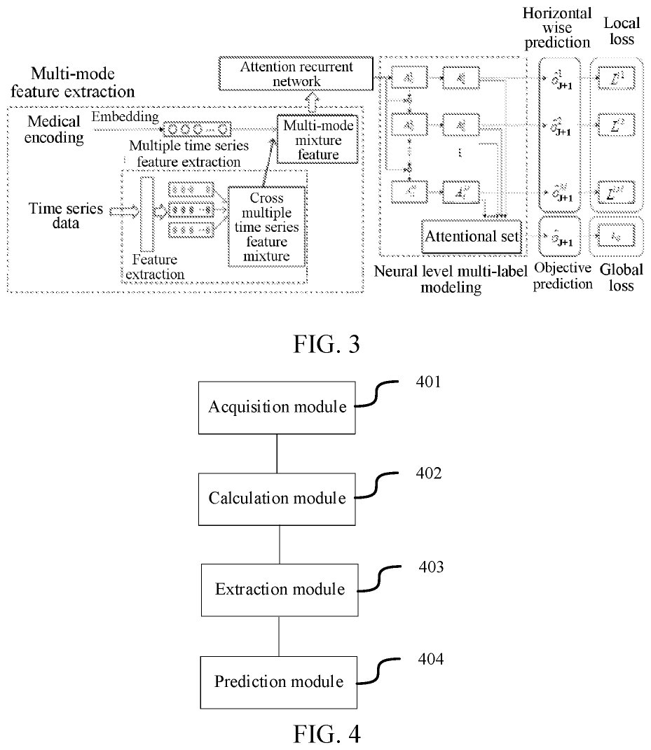

To facilitate understanding of the process shown in step 2011 to step 2014, referring to a multi-mode feature extraction part in FIG. 3, FIG. 3 is a schematic diagram of a diagnosis estimation model according to an embodiment of this disclosure. In the multi-mode feature extraction part, it can be learned that, first, the computer device converts the medical encoded data (that is, the attribute corresponding to the detection data) into a multi-hot vector, and inputs the multi-hot vector into the fully connected neural network. The fully connected neural network may output a feature of the medical encoded data (that is, the feature of the attribute) through calculation. It is to be noted that the process in which the fully connected neural network may output the medical encoded data through calculation is a process of embedding the medical encoded data.

Then, the computer device extracts features from time series data (that is, the detection data) to obtain a feature set. Next, the computer device inputs the feature set into the DCN. The DCN outputs cross multiple time series mixture features, that is, the feature of the detection data. Finally, the computer device mixes the cross multiple time series feature mixture with the feature of the attribute and acquires the multi-mode feature (that is, the detection feature) based on the feature mixture.

It is to be noted that the computer device may obtain the feature of the detection data by using the deep neural network, and may also obtain the feature of the detection data by using a different type of neural network.

202. The computer device inputs the detection feature into a first neural network, and for a detection feature in each time series in the detection feature, the first neural network outputs a first rule feature and a second rule feature different from the first rule feature through two different time series calculations, the first rule feature representing a historical change rule of the detection feature and the second rule feature representing a future change rule of the detection feature.

The first neural network may be a bidirectional recurrent neural network (BiRNN) with an attention mechanism, and the BiRNN may include one first sub-network and one second sub-network, where the first sub-network is configured to acquire the first rule feature and the second sub-network is configured to acquire the second rule feature.

In one implementation, the computer device inputs the detection feature into the first sub-network of the first neural network according to a backward time series sequence, and performs backward time series sequence calculation on the detection feature by using the first subnetwork to obtain the first rule feature; and inputs the detection feature into the second sub-network of the first neural network according to a forward time series sequence, and performs forward time series sequence calculation on the detection feature by using the first sub-network to obtain the second rule feature.

Because at least one sub-detection feature in the detection feature is sorted according to a time sequence, the computer device may input the detection feature into the first sub-network in a forward time series manner, and the computer device may input the detection feature into the second sub-network in a backward time series manner.

In one implementation, the computer device sequentially inputs each sub-detection feature in the detection feature ξ=[ξ₁, ξ₂, . . . ξ_{J}] into a node of an input layer in the first sub-network according to a sequence from rear to front. The computer device inputs ξ_{J} into a first node of the input layer of the first sub-network, inputs ξ_{J-1} into a second node of the input layer of the first sub-network, and so on. After the computer device inputs the detection feature into the first sub-network, the first sub-network may calculate each sub-feature in the detection feature based on a preset calculation rule in the first sub-network, and finally, the first sub-network may output the first rule feature h^{b}=[h₁^{b}, h₂^{b}, . . . h_{J}^{b}], where b is used for representing a backward direction, and the preset calculation rule in the first sub-network is not limited in this embodiment of this disclosure.

In one implementation, the computer device sequentially inputs each sub-detection feature in the detection feature ξ=[ξ₁, ξ₂, . . . ξ_{J}] into a node of an input layer in the second sub-network according to a sequence from front to rear. The computer device inputs ξ₁ into a first node of the input layer of the second sub-network, inputs ξ₂ into a second node of the input layer of the second sub-network, and so on. After the computer device inputs the detection feature into the second sub-network, the second sub-network may calculate each sub-feature in the detection feature based on a preset calculation rule in the second sub-network, and finally, the second sub-network may output the second rule feature h^{f}=[h₁^{f}, h₂^{f}, . . . h_{J}^{f}], where f is used for representing a forward direction, and the preset calculation rule in the second sub-network is not limited in this embodiment of this disclosure.

203. The computer device determines a global feature of the target object based on the first rule feature and the second rule feature.

Because the historical change rule of the detection feature is represented by the first rule feature and the future change rule of the detection feature is represented by the second rule feature, neither of the first rule feature nor the second rule feature can represent a global change rule of the detection feature. To obtain a more accurate global feature of the target object, the computer device may first obtain a rule feature representing the global change rule of the detection feature based on the first rule feature and the second rule feature, and then obtain the global feature according to the rule feature.

In one implementation, step 203 may be implemented through the procedure shown in step 2031 to step 2033.

Step 2031. The computer device splices the first rule feature and the second rule feature to obtain a third rule feature.

After the first sub-network outputs the first rule feature h^{b}=[h₁^{b}, h₂^{b}, . . . h_{J}^{b}] and the second sub-network outputs the second rule feature h^{f}=[h₁^{f}, h₂^{f}, . . . , h_{J}^{f}], the computer device may splice the first rule feature by using the first neural network to obtain the third rule feature h=[h₁, . . . , h_{J}], where hⱼ=[hⱼ^{b},hⱼ^{f}], j is a positive integer, 1≤j≤J, and J represents the total quantity of detection times of the target object.

Step 2032. The computer device weights the third rule feature to obtain a fourth rule feature, the fourth rule feature being used for representing a global change rule of the detection feature.

The computer device may weight the third rule feature by using the attention mechanism in the first neural network. In one implementation, step 2032 may be implemented through the procedure in step 11 to step 13 described below.

Step 11. The computer device performs weight learning based on a first attention mechanism and the third rule feature, to obtain at least one first weight, the first weight being used for representing an importance degree of one detection data and an attribute corresponding to the one detection data.

The first attention mechanism is any attention mechanism in the first neural network, and the computer device may perform weight learning based on a weight learning policy in the first attention mechanism. If the weight learning policy may be a location-based attention weight learning policy, the location-based attention weight learning policy may be expressed as eⱼ^{x}=W_{τ}^{T}hⱼ+bᵣ, where W_{τ} is a second weight vector, W_{τ}^{T} is a transpose matrix of W_{τ}, b_{τ} is a second bias parameter, and eⱼ^{x} is a first weight corresponding to the j^{th} detection.

In this case, the computer device may perform weight learning based on each rule feature in the third rule feature h=[h₁, . . . , h_{J}] and the foregoing location-based attention weight learning policy, to obtain J first weights, the J first weights being also the at least one first weight.

It is to be noted that the weight learning policy in the first attention mechanism may alternatively be another attention weight learning policy, and the weight learning policy in the first attention mechanism is not limited in this embodiment of this disclosure.

Step 12. The computer device normalizes the at least one first weight to obtain at least one second weight.

Because the first weight in step 11 is a value obtained through mathematical calculation, the at least one first weight may be excessively large or excessively small. For ease of calculation, the at least one first weight may be normalized, so that each second weight obtained after processing is moderate. In this case, when the at least one first weight is excessively large, the at least one first weight may be reduced proportionally, and when the at least one first weight is excessively small, the at least one first weight may be enlarged proportionally, to normalize the at least one first weight.

Because each second weight is a result of normalizing one first weight, the second weight has the similar function as the first weight, and both are used for representing an importance degree of one detection data and an attribute corresponding to the one detection data.

Step 13. The computer device weights the third rule feature based on the at least one second weight, to obtain a fourth rule feature.

The computer device substitutes the at least one second weight [α₁^{x}, . . . , αⱼ^{x} . . . , α_{J}^{x}] into a third equation, and uses an output of the third equation as the fourth rule feature to weight the third rule feature. The third equation may be expressed as c=Σⱼ₌₁^{J}αⱼ^{x}hⱼ, where c is the fourth rule feature, αⱼ^{x} is a second weight corresponding to the j^{th} detection, and J is the total quantity of detection times of the target object.

Because the second weight is used for representing the importance degree of one detection data and the attribute corresponding to the one detection data, the third rule feature is expressed more intensively by weighting the third rule feature by using the at least one second weight. Therefore, the fourth rule feature may represent the global change rule of the detection feature.

Because the first rule feature and the second rule feature are weighted, the first rule feature and the second rule feature may be integrally represented by the fourth rule feature, so that the fourth rule feature can not only represent the historical change rule expressed by the first rule feature, but also represent the future change rule represented by the first rule feature. Therefore, the fourth rule feature may represent the global change rule of the detection feature.

Step 2033. The computer device determines the global feature based on the third rule feature and the fourth rule feature.

Because a correlation between adjacent detection data and a correlation between adjacent attributes are the highest, to further predict an attribute of the target object during next detection, the computer device may substitute a rule feature corresponding to a last detection time in the third rule feature and the fourth rule feature into a fourth equation, and use an output of the fourth equation as the global feature, where the fourth equation may be expressed as ĉ=ReLU(W_{d}^{T}[h_{J},c]+b_{d}), where ĉ is the global feature, h_{J} is the rule feature corresponding to the last detection time of the target object in the third rule feature, [[h_{J},c] is a vector obtained after h_{J} and c are spliced, W_{d} is a third weight matrix, W_{d}^{T} is a transposed matrix of W_{d}, b_{d} is a third bias parameter, and ReLU( ) refers to a rectified linear unit function.

Because the fourth rule feature represents the global change rule of the detection feature, the first rule feature may represent the historical change rule of the detection feature, and the second rule feature may represent the future change rule of the detection feature, a result obtained by weighting the three rule features may represent the global feature of the target object.

204. The computer device inputs the global feature into a second neural network; and the second neural network extracts and outputs at least one local feature of the target object from the global feature.

The second neural network may be a hierarchical multi-label classification network (HMCN), and the second neural network may extract a local feature from the inputted global feature. Because the global feature cannot represent details of the target object, the details of the target object may be extracted by using the second neural network. The second neural network may extract the details of the target object step by step, so that the finally extracted details can meet requirements of attribute prediction.

Each layer of the second neural network may output one of the local features. After the computer device inputs the global feature into the second neural network, the global feature may be inputted from the input layer to an output layer of the second neural network. The second neural network may calculate the global feature layer by layer. During calculation, a first target layer of the second neural network may calculate a hierarchical feature of the first target layer and a local feature of the target object in the first target layer based on output data of the second target layer, where the first target layer is any layer of the second neural network and the second target layer is an upper layer of the first target layer in the second neural network. The hierarchical feature is used for representing a state of the global feature in a network layer of the second neural network, and the hierarchical feature of the first target layer is determined by the global feature and a hierarchical feature of the second target layer.

After the second target layer of the second neural network generates the hierarchical feature of the second target layer and a local feature of the target object in the second target layer, the second target layer may output the hierarchical feature of the second target layer and the global feature (the output data of the second target layer) to the first target layer, so that the first target layer may receive the hierarchical feature of the second target layer and the global feature. In this case, with the global features outputted by each network layer of the second neural network, the global feature may be inputted to each network layer of the second neural network.

Because the hierarchical feature of the first target layer is determined by the global feature and the hierarchical feature of the second target layer, after the first target layer receives the hierarchical feature of the second target layer and the global feature, the first target layer may calculate the hierarchical feature of the first target layer based on the hierarchical feature of the second target layer and the global feature. In one implementation, a hierarchical feature of an i^{th} layer of the second neural network may be expressed as A_{G}ⁱ=ReLU(W_{G}ⁱ(A_{G}ⁱ⁻¹+ĉ)+b_{G}), where G represents global, W_{G}ⁱ is a fourth weight matrix, A_{G}ⁱ⁻¹ is a hierarchical feature of an (i−1)^{th} layer, A_{G}ⁱ represents the hierarchical feature of the i^{th} layer, b_{G} is a fourth bias parameter, and the i^{th} layer may be deemed as the first target layer.

A node in the first target layer may acquire a local feature of the target object in the first target layer based on the hierarchical feature of the first target layer and the global feature. In one implementation, a local feature A_{L}ⁱ of the target object in the i^{th} layer may be expressed as A_{L}ⁱ=ReLU(W_{T}ⁱA_{G}ⁱ+b_{T}), where L represents a network layer, W_{T}ⁱ is a fifth weight matrix, and b_{T} is a fifth bias parameter.

Because each layer of the second neural network performs calculation based on the hierarchical feature of the upper layer and the global feature, the local feature of the target object in each layer of the second neural network is affected by the local feature of the upper layer. Because hierarchical expression of each layer is determined by a hierarchical feature of this layer, a local feature generated by any network layer in the second neural network may be used as a parent of a local feature generated by a next network layer. Therefore, the second neural network may extract the details of the target object step by step.

205. The computer device predicts an attribute of the target object based on the at least one local feature of the target object.

Because one local feature may represent different levels of details of the target object, considering that there are many details, the details may be processed centrally to obtain a more detailed local feature, and attribute prediction is then performed according to the more detailed local feature.

In one implementation, step 205 may be implemented through the procedure shown in step 2051 and step 2052.

Step 2051. The computer device weights the at least one local feature of the target object to obtain a target local feature.

The target local feature is also the more detailed local feature. The computer device may weight the local feature of the target object by using the attention mechanism in the second neural network. In one implementation, step 2051 may be implemented through the procedure shown in step 21 and step 22.

Step 21. The computer device performs weight learning based on the second attention mechanism and the at least one local feature, to obtain at least one third weight, one third weight being used for representing an importance degree of one local feature.

The second attention mechanism is any attention mechanism in the second neural network, and the computer device performs weight learning based on the second attention mechanism and the at least one local feature, and may learn weights by using a weight learning policy in the second attention mechanism. The weight learning policy in the second attention mechanism may be expressed as:

$${α}_{i}^{y} = \frac{exp ( {e}_{i}^{y} )}{{\sum }_{j = 1 , … , M} exp ( {e}_{j}^{y} )} , {e}_{i}^{y} = tanh ( {W}_{α} {A}_{L}^{i} + {b}_{α} ) , {e}_{i}^{y} ∈ [ {e}_{1}^{y} , … , {e}_{M}^{y} ]$$

where αᵢ^{y} is an i^{th} third weight, W_{α} is a sixth weight matrix, b_{α} is a sixth bias parameter, eⱼ^{y} is a parameter weight corresponding to the j^{th} detection time, and M represents a quantity of local features.

It is to be noted that the weight learning policy in the second attention mechanism may alternatively be another weight learning policy, and the weight learning policy in the second attention mechanism is not limited in this embodiment of this disclosure.

Step 22. The computer device weights the at least one local feature of the target object based on the at least one third weight, to obtain the target local feature.

The computer device substitutes at least one third weight and the at least one local feature of the target object into a fifth equation, and uses an output of the fifth equation as the target local feature to weight the at least one local feature of the target object. The fifth equation may be expressed as:

$${A}_{G} = {\sum }_{i = 1 , … N} {α}_{i} {A}_{L}^{i}$$

where A_{G} is an attribute corresponding to detection data that is corresponding to a current time series and N is a quantity of layers of the second neural network.

Because one third weight represents an importance degree of one local feature, the obtained target local feature is more detailed by weighting the at least one local feature by using the at least one third weight.

Step 2052. Predict an attribute of the target object based on the target local feature.

The computer device may substitute the target local feature into a sixth equation to predict the attribute of the target object. The sixth equation is used for predicting the attribute of the target object. The sixth equation may be expressed as ô_{J+1}=Sigmoid(W_{G}A_{G}+b_{G}), where ô_{J+1} is a predicted attribute of the target object corresponding to (J+1)^{th} data detection, W_{G} is a seventh weight matrix, and b_{G} is a seventh bias parameter.

In some embodiments, the second neural network may determine whether to output a currently predicted attribute according to a global loss and a local loss in the second neural network.

In some possible implementations, after the global feature is inputted to the second neural network, if the global loss and the local loss in the second neural network meet a preset condition, the second neural network outputs the currently predicted attribute; otherwise, the second neural network adjusts a weight matrix in the second neural network until the global loss and the local loss in the second neural network meet the preset condition. The local loss is a difference between expected output data and actual output data in each layer of the second neural network, and the global loss is a difference between expected final output data and actual final data of the second neural network.

After the calculation of any layer of the second neural network is finished, the second neural network may predict a local feature of any layer at a next detection time (referred to as a predicted local feature for short). In this case, a predicted local feature ô_{J+1}ⁱ of the i^{th} layer may be expressed as:

ô_{J+1}ⁱ=Sigmoid(W_{L}ⁱA_{L}ⁱ+b_{L})

where W_{L}ⁱ is an eighth weight matrix of the i^{th} layer, and b_{L} is an eighth bias parameter.

The second neural network may predict an attribute of at least one target object. When predicting the attribute of the at least one target object based on the second neural network, the second neural network may calculate a local loss of any layer based on a predicted local feature of any layer and by using a cross entropy policy. In this case, a local loss L^{li} of the i^{th} layer may be expressed as:

$${L}^{li} = - \frac{1}{Q} {\sum }_{i = 1}^{N} {o}_{J + 1}^{( i , Q )} log ( {\hat{o}}_{J + 1}^{( i , Q )} ) + ( 1 - {o}_{J + 1}^{( i , Q )} ) log ( 1 - {\hat{o}}_{J + 1}^{( i , Q )} )$$

where Q is quantity of target objects, o_{J+1}^{(i,Q)} is actual output data of the i^{th} layer based on a global feature of a Q^{th} target object, and ô_{J+1}^{(i,Q)} is predicted output data of the i^{th} layer based on the global feature of the Q^{th} target object.

When predicting the attribute of the at least one target object based on the second neural network, if the calculation of each layer of the second neural network is finished, the second neural network may calculate the attribute of the at least one target object during next detection, and the second neural network may then calculate a global loss L^{G} based on the predicted attribute of the at least one target object during next detection by using the cross entropy policy, where L^{G} may be expressed as:

$${L}^{G} = - \frac{1}{Q} {\sum }_{i = 1}^{N} {o}_{J + 1}^{Q} log ( {\hat{o}}_{J + 1}^{Q} ) + ( 1 - {o}_{J + 1}^{Q} ) log ( 1 - {\hat{o}}_{J + 1}^{Q} )$$

where o_{J+1}^{Q} is an actually outputted attribute of the Q^{th} target object during next detection, and ô_{J+1}^{Q} is a predicted attribute of the Q^{th} target object during next detection.

The preset condition may be expressed as Loss=L^{G}+γ(L^{l1}+L^{l2} . . . L^{lp}), where p is an integer greater than 2, for example, p=3, Loss is a preset convergence value and γ is a predefined parameter, and is used for balancing the global loss and the local loss. When the computer device inputs Loss, γ, L^{l1}, L^{l2}, . . . , and L^{lp} into the equation of the preset condition, if the above equation holds, the global loss and the local loss in the second neural network meet the preset condition; otherwise, the global loss and the local loss in the second neural network do not meet the preset condition.

When the global loss and the local loss in the second neural network meet the preset condition, it indicates that a difference between the generated local feature and the expected local feature of each layer of the second neural network reaches preset precision, thereby ensuring relatively high precision of the local feature of each layer of the second neural network and further improving the precision of the predicted attribute.

It is to be noted that because the second neural network performs calculation based on values, and each state in the attribute of the target object is actually represented by a state identifier, the computer device further needs to convert the data outputted by the second neural network into an attribute composed of the state identifier. The data outputted by the second neural network may include at least one probability value, and each probability value corresponds to a state in the attribute of the target object. When any probability value is greater than a target value, and it indicates that the target object has a target state corresponding to the any probability, the computer device stores the first state identifier in a position of the target state in the attribute. When any probability value is less than or equal to the target value, and it indicates that the target object does not have the target state corresponding to the any probability, the computer device stores the second state identifier in the position of the target state in the attribute. In this case, an actual expression of the attribute can be obtained by determining each probability value, and the target value is not limited in this embodiment of this disclosure.

To further describe the process shown in step 203 and step 204, refer to the neural level multi-label modeling part in FIG. 3. From this part, it can be learned that output data (that is, the global feature) of an attention recurrent network goes to the neural level multi-label modeling part, and the attention recurrent network is equivalent to the first neural network. The computer device inputs the global feature into each layer of the second neural network in the neural level multi-label modeling part. The first layer generates a hierarchical feature A_{G}¹ of the first layer according to the global feature, then generates a local feature A_{L}¹ of the first layer according to A_{G}¹, and may perform data prediction based on A_{L}¹ to obtain output data ô_{J+1}¹ predicted by the first layer. The computer device calculates a local loss L^{l1} of the first layer, and the first layer outputs A_{G}¹ to the second layer, so that the second layer may perform a calculation process similar to that of the first layer. Finally, all layers of the second neural network may get one A_{L}ⁱ. The computer device outputs M A_{L}ⁱ to an attentional ensemble. In the attention set and based on the second attention mechanism, the computer device generates predicted output data ô_{J+1} and further generates global loss L_{G} according to the predicted output data ô_{J+1}. In this case, when both the global loss L_{G} and the local losses Lᵢ both meet the present condition, the second neural network may output ô_{J+1}.

In the method for predicting an attribute of a target object based on machine learning provided in the embodiments of this disclosure, the global feature of the target object is determined based on the rule feature representing the historical and future change rules of the detection feature, and the global feature is refined to obtain at least one local feature of the target object, so that the refined local feature can better reflect the feature of the target object, and the attribute of the target object is further predicted based on the local feature. Therefore, the precision of the predicted attribute can be improved. When the attribute of the target object is a predicted diagnosis result, the precision of the predicted diagnosis result can be improved. In addition, because the detection feature is obtained based on the detection data and the attribute corresponding to the detection data, compared with the feature of the target object obtained only based on the detection data, the detection feature in this embodiment of this disclosure can better reflect the feature of the target object during detection. Moreover, the detection data is actually detected data, and may be used as an objective basis, so that the obtained detection feature is interpretable, and the attribute corresponding to the detection data is a result of a subjective judgment, so that the precision of the detection feature obtained based on the attribute and the detection data is relatively high. Furthermore, when the global loss and the local loss in the second neural network meet the preset condition, it indicates that the local feature generated by each layer of the second neural network reaches an expected value, thereby ensuring relatively high precision of the local feature outputted by the output layer of the second neural network.

FIG. 4 is a schematic structural diagram of an apparatus for predicting an attribute of a target object based on machine learning according to an embodiment of this disclosure. The apparatus includes:

an acquisition module 401, configured to determine a detection feature of the target object according to detection data of the target object and an attribute corresponding to the detection data;

a calculation module 402, configured to input the detection feature into a first neural network; and for a detection feature in each time series in the detection feature, output, by the first neural network, a first rule feature and a second rule feature different from the first rule feature through two different time series calculations, the first rule feature representing a historical change rule of the detection feature and the second rule feature representing a future change rule of the detection feature;

the acquisition module 401 being further configured to determine a global feature of the target object based on the first rule feature and the second rule feature;

an extraction module 403, configured to input the global feature into a second neural network; and extract and output, by the second neural network, at least one local feature of the target object from the global feature; and

a prediction module 404, configured to predict the attribute of the target object based on the at least one local feature of the target object.

In some embodiments, the acquiring module 401 is configured to:

input the attribute corresponding to the detection data into a fully connected neural network, delete an unessential factor in the attribute corresponding to the detection data by using the fully connected neural network to obtain a feature of the attribute corresponding to the detection data;

input the detection data into a time series analysis tool, extract a feature of each type of data in the detection data in each time series by using the time series analysis tool, and output a feature set;

input the feature set into a deep & cross neural network, and perform cross processing on a feature of each time series in the feature set by using the deep & cross neural network to obtain a feature of the detection data; and

input the feature of the attribute and the feature of the detection data into a deep neural network, extract a mixture feature of the detection data and the attribute corresponding to the detection data by using the deep neural network, and output the detection feature.

In some embodiments, the acquiring module 401 is configured to:

splice the first rule feature and the second rule feature to obtain a third rule feature;

weight the third rule feature to obtain a fourth rule feature, the fourth rule feature being used for representing a global change rule of the detection feature; and

determine the global feature based on the third rule feature and the fourth rule feature.

In some embodiments, the acquiring module 401 may be configured to:

perform weight learning based on a first attention mechanism and the third rule feature, to obtain at least one first weight, the one first weight being used for representing an importance degree of one detection data and an attribute corresponding to the one detection data;

normalize the at least one first weight to obtain at least one second weight; and

weight the third rule feature based on the at least one second weight, to obtain the fourth rule feature.

In some embodiments, the prediction module 404 includes:

a processing unit, configured to weight at least one local feature of the target object to obtain a target local feature; and

a prediction unit, configured to predict the attribute of the target object based on the target local feature.

In some embodiments, the processing unit is configured to:

perform weight learning based on a second attention mechanism and the at least one local feature, to obtain at least one third weight, one third weight being used for representing an importance degree of one local feature; and

weight the at least one local feature based on the at least one third weight to obtain the target local feature.

In some embodiments, each layer of the second neural network outputs one of the local features.

In some embodiments, the apparatus further includes an output module, configured to, after the global feature is inputted to the second neural network, in a case that a global loss and a local loss in the second neural network meet a preset condition, output, by the second neural network, a currently predicted attribute, the local loss being a difference between expected output data and actual output data in each layer of the second neural network, and the global loss being a difference between expected final output data and actual final data of the second neural network.

In some embodiments, the apparatus further includes a generating module, configured to generate, based on a hierarchical feature of a first target layer and a local feature generated by a second target layer in the second neural network, a local feature outputted by the first target layer, the hierarchical feature of the first target layer being used for representing a state of the global feature in the first target layer, and the second target layer being an upper layer of the first target layer in the second neural network.

In some embodiments, the hierarchical feature of the first target layer is determined by the global feature and a hierarchical feature of the second target layer.

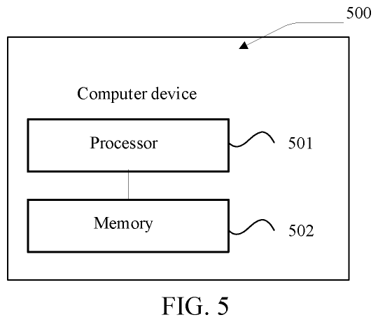

FIG. 5 is a schematic structural diagram of a computer device according to an embodiment of this disclosure. The computer device 500 may vary greatly due to different configurations or performances, and may include one or more central processing units (CPU) 501 and one or more memories 502, where the memory 502 stores at least one instruction, and the at least one instruction is loaded performed by the processor 501 to implement the method for predicting an attribute of a target object based on machine learning according to the foregoing method embodiments. Certainly, the computer device 500 may further include components such as a wired or wireless network interface, a keyboard, and an input/output (I/O) interface, to facilitate inputs and outputs. The computer device 500 may further include another component configured to implement a function of a device. Details are not further described herein.

In an exemplary embodiment, a non-transitory computer-readable storage medium, for example, a memory including instructions, is further provided. The instructions may be executed by the processor in the terminal to implement the method for predicting an attribute of a target object based on machine learning in the foregoing embodiments. For example, the computer-readable storage medium may be a read-only memory (ROM), a random access memory (RAM), a compact disc read-only memory (CD-ROM), a magnetic tape, a floppy disk, an optical data storage device, or the like.

In an exemplary embodiment, a computer program product including instructions is further provided, the instructions, when run on a computer, causing the computer to perform the method for predicting an attribute of a target object based on machine learning in the foregoing embodiments.

Any combination of the foregoing exemplary technical solutions may be used to form another embodiment of the present disclosure. Details are not described herein again.

It is to be noted that when the apparatus for predicting an attribute of a target object based on machine learning according to the foregoing embodiments predicts the attribute, only divisions of the foregoing functional modules are described by using an example. During actual application, the foregoing functions may be allocated to and completed by different functional modules according to requirements, that is, the internal structure of the apparatus is divided into different functional modules, to complete all or some of the foregoing described functions. In addition, the apparatus for predicting an attribute of a target object based on machine learning provided in the foregoing embodiments belongs to the same concept as the embodiments of the method for predicting an attribute of a target object based on machine learning. For the specific implementation process, refer to the method embodiments, and details are not described herein again.

The term module (and other similar terms such as unit, submodule, etc.) in this disclosure may refer to a software module, a hardware module, or a combination thereof. A software module (e.g., computer program) may be developed using a computer programming language. A hardware module may be implemented using processing circuitry and/or memory. Each module can be implemented using one or more processors (or processors and memory). Likewise, a processor (or processors and memory) can be used to implement one or more modules. Moreover, each module can be part of an overall module that includes the functionalities of the module.

A person of ordinary skill in the art may understand that all or some of the steps of the foregoing embodiments may be implemented by hardware, or may be implemented a program instructing related hardware. The program may be stored in a non-transitory computer-readable storage medium. The storage medium may be: a ROM, a magnetic disk, or an optical disc.

The foregoing descriptions are merely exemplary embodiments of this disclosure, but are not intended to limit this application. Any modification, equivalent replacement, or improvement and the like made within the spirit and principle of this application fall within the protection scope of this application.

What is claimed is:

1. A method for predicting an attribute of a target object based on machine learning, performed by a computer device, the method comprising:
determining detection features of the target object according to detection data of the target object and an attribute corresponding to the detection data;
inputting the detection features into a first neural network;
for a detection feature in each time series in the detection features, outputting, by the first neural network, a first rule feature and a second rule feature different from the first rule feature through two different time series calculations, the first rule feature representing a historical change rule of the detection feature and the second rule feature representing a future change rule of the detection feature;
determining a global feature of the target object based on the first rule feature and the second rule feature;
inputting the global feature into a second neural network;
extracting and outputting, by the second neural network, at least one local feature of the target object from the global feature; and
predicting the attribute of the target object based on the at least one local feature of the target object.

2. The method according to claim 1, wherein determining the detection features of the target object according to the detection data of the target object and the attribute corresponding to the detection data comprises:
inputting the attribute corresponding to the detection data into a fully connected neural network, to screen out a target state in the attribute by using the fully connected neural network, to weight the target state, and to output a feature of the attribute based on the weighted target state;
inputting the detection data into a time series analysis tool, to extract a feature of each type of data in the detection data in each time series by using the time series analysis tool, and to output a feature set;
inputting the feature set into a deep & cross neural network, to perform cross processing on a feature of each time series in the feature set and to obtain a feature of the detection data; and
inputting the feature of the attribute and the feature of the detection data into a deep neural network, to extract a mixture feature of the detection data and the attribute corresponding to the detection data, and to output the mixture feature as the detection feature.

3. The method according to claim 1, wherein determining, based on the first rule feature and the second rule feature, the global feature of the target object comprises:
splicing the first rule feature and the second rule feature to obtain a third rule feature;
weighting the third rule feature to obtain a fourth rule feature, the fourth rule feature being used for representing a global change rule of the detection feature; and
determining the global feature based on the third rule feature and the fourth rule feature.

4. The method according to claim 3, wherein weighting the third rule feature to obtain the fourth rule feature comprises:
performing weight learning based on a first attention mechanism and the third rule feature, to obtain at least one first weight, the at least one first weight being used for representing an importance degree of the detection data and an attribute corresponding to the detection data;
normalizing the at least one first weight to obtain at least one second weight; and
weighting the third rule feature based on the at least one second weight, to obtain the fourth rule feature.

5. The method according to claim 1, wherein predicting the attribute of the target object based on the at least one local feature of the target object comprises:
weighting the at least one local feature of the target object to obtain a target local feature; and
predicting the attribute of the target object based on the target local feature.

6. The method according to claim 5, wherein weighting the at least one local feature of the target object to obtain the target local feature comprises:
performing weight learning based on a second attention mechanism and the at least one local feature, to obtain at least one third weight, the at least one third weight being used for representing an importance degree of the at least one local feature; and
weighting the at least one local feature based on the at least one third weight, to obtain the target local feature.

7. The method according to claim 1, wherein each layer of the second neural network outputs one of the at least one local feature.

8. The method according to claim 1, further comprising:
after the global feature is inputted to the second neural network, in response to a global loss and a local loss in the second neural network meeting a preset condition, outputting, by the second neural network, a currently predicted attribute, the local loss being a difference between expected output data and actual output data in each layer of the second neural network, and the global loss being a difference between expected final output data and actual final output data of the second neural network.

9. The method according to claim 1, further comprising:
generating, based on a hierarchical feature of a first target layer and a local feature generated by a second target layer in the second neural network, a local feature outputted by the first target layer, the hierarchical feature of the first target layer being used for representing a state of the global feature in the first target layer, and the second target layer being an upper layer of the first target layer in the second neural network.

10. The method according to claim 9, wherein the hierarchical feature of the first target layer is determined by the global feature and a hierarchical feature of the second target layer.

11. A device for predicting an attribute of a target object based on machine learning, comprising a memory for storing instructions and a processor in communication with the memory, wherein the processor is configured to execute the instructions to cause the device to:
determine detection features of the target object according to detection data of the target object and an attribute corresponding to the detection data;
input the detection features into a first neural network;
for a detection feature in each time series in the detection features, output, by the first neural network, a first rule feature and a second rule feature different from the first rule feature through two different time series calculations, the first rule feature representing a historical change rule of the detection feature and the second rule feature representing a future change rule of the detection feature;
determine a global feature of the target object based on the first rule feature and the second rule feature;
input the global feature into a second neural network;
extract and output, by the second neural network, at least one local feature of the target object from the global feature; and
predict the attribute of the target object based on the at least one local feature of the target object.

12. The device according to claim 11, wherein the processor, when executing the instructions to cause the device to determine the detection features of the target object according to the detection data of the target object and the attribute corresponding to the detection data, is configured to cause the device to:
input the attribute corresponding to the detection data into a fully connected neural network, to screen out a target state in the attribute by using the fully connected neural network, to weight the target state, and to output a feature of the attribute based on the weighted target state;
input the detection data into a time series analysis tool, to extract a feature of each type of data in the detection data in each time series by using the time series analysis tool, and to output a feature set;
input the feature set into a deep & cross neural network, to perform cross processing on a feature of each time series in the feature set, and to obtain a feature of the detection data; and
input the feature of the attribute and the feature of the detection data into a deep neural network, to extract a mixture feature of the detection data and the attribute corresponding to the detection data, and to output the mixture feature as the detection feature.

13. The device according to claim 11, wherein the processor, when executing the instructions to cause the device to determine, based on the first rule feature and the second rule feature, the global feature of the target object, is configured to cause the device to:
splice the first rule feature and the second rule feature to obtain a third rule feature;
weight the third rule feature to obtain a fourth rule feature, the fourth rule feature being used for representing a global change rule of the detection feature; and
determine the global feature based on the third rule feature and the fourth rule feature.

14. The device according to claim 13, wherein the processor, when executing the instructions to cause the device to weight the third rule feature to obtain the fourth rule feature, is configured to cause the device to:
perform weight learning based on a first attention mechanism and the third rule feature, to obtain at least one first weight, the at least one first weight being used for representing an importance degree of the detection data and an attribute corresponding to the detection data;
normalize the at least one first weight to obtain at least one second weight; and
weight the third rule feature based on the at least one second weight, to obtain the fourth rule feature.

15. The device according to claim 11, wherein the processor, when executing the instructions to cause the device to predict the attribute of the target object based on the at least one local feature of the target object, is configured to cause the device to:
weight the at least one local feature of the target object to obtain a target local feature; and
predict the attribute of the target object based on the target local feature.

16. The device according to claim 15, wherein the processor, when executing the instructions to cause the device to weighting the at least one local feature of the target object to obtain the target local feature, is configured to cause the device to:
perform weight learning based on a second attention mechanism and the at least one local feature, to obtain at least one third weight, the at least one third weight being used for representing an importance degree of the at least one local feature; and
weight the at least one local feature based on the at least one third weight, to obtain the target local feature.

17. The device according to claim 11, wherein each layer of the second neural network outputs one of the at least one local feature.

18. The device according to claim 11, wherein, when the processor executes the instructions, the processor is configured to further cause the device to:
after the global feature is inputted to the second neural network, in response to a global loss and a local loss in the second neural network meeting a preset condition, output, by the second neural network, a currently predicted attribute, the local loss being a difference between expected output data and actual output data in each layer of the second neural network, and the global loss being a difference between expected final output data and actual final output data of the second neural network.

19. The device according to claim 11, wherein, when the processor executes the instructions, the processor is configured to further cause the device to:
generate, based on a hierarchical feature of a first target layer and a local feature generated by a second target layer in the second neural network, a local feature outputted by the first target layer, the hierarchical feature of the first target layer being used for representing a state of the global feature in the first target layer, and the second target layer being an upper layer of the first target layer in the second neural network.

20. A non-transitory storage medium for storing computer readable instructions, the computer readable instructions, when executed by a processor to predict an attribute of a target object based on machine learning, causing the processor to:
determine detection features of the target object according to detection data of the target object and an attribute corresponding to the detection data;
input the detection features into a first neural network;
for a detection feature in each time series in the detection features, output, by the first neural network, a first rule feature and a second rule feature different from the first rule feature through two different time series calculations, the first rule feature representing a historical change rule of the detection feature and the second rule feature representing a future change rule of the detection feature;
determine a global feature of the target object based on the first rule feature and the second rule feature;
input the global feature into a second neural network;
extract and output, by the second neural network, at least one local feature of the target object from the global feature; and
predict the attribute of the target object based on the at least one local feature of the target object.
